(19) United States
(12) Patent Application Publication
(10) Pub. No.: US 2006/0141492 A1
(43) Pub. Date: Jun. 29, 2006

(54) Gene probes for the selective detection of microorganisms that reductively dechlorinate polychlorinated biphenyl compounds

(76) Inventors: Kevin R. Sowers, Baltimore, MD (US); Sonja K. Fagervoid, Baltimore, MD (US); Joy E.M. Watts, Baltimore, MD (US); Harold D. May, Pleasant, SC (US)

Correspondence Address: INTELLECTUAL PROPERTY / TECHNOLOGY LAW, PO BOX 14329, RESEARCH TRIANGLE PARK, NC, 27709, US

(21) Appl. No.: 11/190,801

(22) Filed: Jul. 27, 2005

Related U.S. Application Data
(60) Provisional application No. 60/591,514, filed on Jul. 27, 2004.

(51) Int. Cl. C12Q 1/68; C07H 21/04; C12N 1/20; B09C 1/10

(52) U.S. Cl. 435/6; 435/252.3; 536/24.1; 435/262.5

(57) ABSTRACT

The present invention relates to an assay for identification of PCB dechlorinating organisms that are capable of biologically removing PCBs from contaminated materials. Specifically, the invention provides a set of primers for detecting PCB dechlorinating organisms in a sample. These individual primers of the primer set have a sequence of at least 12 nucleotides that is unique to 16S rDNA of PCB dechlorinating organisms.

### CROSS-REFERENCE TO RELATED APPLICATIONS

This application claims priority from U.S. Provisional Patent Application No. 60/591,514 filed on Jul. 27, 2004 in the names of Kevin R. Sowers, Joy E. M. Watts, Sonja K. Fagervold and Harold D. May for “GENE PROBES FOR THE SELECTIVE DETECTION OF MICROORGANISMS THAT REDUCTIVELY DECHLORINATE POLYCHLORINATED BIPHENYL COMPOUNDS,” the contents of which are incorporated by reference herein for all purposes.

### BACKGROUND OF THE INVENTION

1. Field of the Invention

The present invention relates to a method and use of probes or primers for identifying PCB dechlorinating microorganisms which are effective in dechlorinating PCB mixtures containing widely varying and significant numbers of PCB congeners.

2. Description of the Related Art

Polychlorinated biphenyls (PCBs) are haloaromatic compounds having exceptional chemical stability. Environmental and toxicological problems caused by the use of PCBs have resulted in restriction of their production under the Toxic Substances Control Act of 1976 and a complete ban of their manufacture by the United States Environmental Protection Agency in 1979. Past disposal practices have resulted in substantial PCB contamination of soils and surface water sediments. Consequently, in the United States, at least 15% of the PCBs manufactured to date remains in the environment as a highly recalcitrant contaminant. Acute toxicological effects of PCB exposure include chloracne (a skin disease), teratotoxicity, endocrine effects, immunotoxicity, carcinogenicity, and hepatotoxicity (liver damage) (35, 37). The mutagenic and carcinogenic character of PCBs and their suspected role in the reproductive failure of wildlife species are issues of great concern. Further, these compounds bioaccumulate and biomagnify in the fatty tissue of animals in the food web, such as fish, which can affect the human population because of food consumption. In sum, the toxicological findings on PCBs and their propensity for bioaccumulation raise concern for the well being of both humans and wildlife.

Historically, harbor regions have been heavily impacted by the accumulation of polychlorinated biphenyls due to their use in and inadvertent release from naval and industrial applications. Due to their hydrophobic character, PCBs strongly associate with organic carbon, clays and silt that settle into the anaerobic regions of marine sediments. Reports of the distribution of PCBs in marine coastal harbor regions (e.g. Baltimore Harbor, New Bedford Harbor, Charleston Harbor, Newark Bay, and Los Angeles Harbor among others) demonstrate the tenacity of PCB contamination (4, 20, 24, 38).

In aerobic environments, PCBs undergo microbial degradation with oxygen addition at the 2, 3 positions by a dioxygenase and subsequent dehydration to catechol followed by ring cleavage. Although lesser chlorinated PCBs ranging from mono- to hexa-chlorinated congeners can be degraded aerobically, extensively chlorinated congeners (e.g., tetrasubstituted) such as those prominent in Aroclor 1260, a formerly commonly used PCB material, are not transformed under aerobic conditions. In this respect, most aerobic degradative activity is restricted to congeners with less than 4 to 6 chlorines, depending on the positions of the chloro substituents on the rings. This is a small region of the structural spectrum of PCBs, since there are 209 congeners (isomers and homologs) of PCBs. Commercial mixtures of PCBs formerly marketed in the United States under the Aroclor trademark typically contained more than 50 of such congeners. The extent of chlorination of the PCBs varies with the specific commercial mixture. For example, Aroclor 1242 is dominated by tri- and tetrachlorobiphenyls, the aforementioned Aroclor 1260 is dominated by penta-, hexa- and heptachlorobiphenyls, and Aroclor 1268 is dominated by hepta-, octa- and nanachlorobiphenyls. Even less-chlorinated Aroclors contain significant levels of congeners with 5 or more chlorine substituents. For this reason, even a consortium of aerobic bacteria (a consortium being a population of bacteria containing different strains with different congener (degradative) & specificity) cannot remove Aroclor PCB compositions from the environment.

PCBs accumulate in the anaerobic zone of marine and estuarine sediments and therefore serve as reservoirs of PCB. Anaerobic dechlorination of PCBs is a critical step in the biodegradation of these anthropogenic compounds in anaerobic sediments. Aerobic degradation involves biphenyl ring cleavage, but within anaerobic sediments the microbial reductive dehalogenation results in the sequential removal of chlorine atoms from the biphenyl ring (5, 7). The present inventors, in U.S. patent application Ser. No. 09/860,200, identified for the first time two PCB dechlorinating bacteria that have been designated as bacterium double flank-dechlorinating strain (DF-1) and bacterium ortho-dechlorinating stain (0-17), within the green non-sulfur Chloroflexi phylum. Both of these microorganisms couple their growth to the reductive dechlorination of PCB (14, 32, 43, 45). Fennel and co-workers (18) reported that another species within the Chloroflexi, Dehalococcoides ethenogenes 195, co-metabolically dechlorinated the PCB 2,3,4,5,6-pentachlorobiphenyl and other aromatic organochlorines when grown with tetrachloroethene. This microorganism was the first species to be isolated and described in the Dehalococcoides group (28). Other Dehalococcoides spp. use chlorinated ethenes as electron acceptors including strains VS (12), FL2 (27), BAV1 (21), CBDB1 (2) and KB-1/VC-H₂ (15). In addition to chlorinated ethenes, strain CBDB1 dechlorinates chlorinated benzenes and dioxins (1, 8). Little is known about the distribution and catalytic diversity of PCB dechlorinating bacteria, particularly because they appear to be a small part of microbial communities in the environment and are difficult to detect using universal 16S rRNA gene PCR primers (43).

U.S. Patent Application No. 20030077601 identifies 16S rRNA regions from Dehalococcoides ethenogenes and other bacteria that are capable of reductive dechlorination that enable the identification of dechlorinating bacterial organisms. Probes and primers corresponding to the unique regions have been constructed to enable the rapid identification of the dechlorinators. However, the primers described in U.S. Patent Application No. 20030077601 have not been found to be effective in determining the PCB dechlorinators.

Thus, in order to completely biodegrade PCBs, both anaerobic and aerobic microorganisms are required since the anaerobic microorganisms dechlorinate more extensively chlorinated PCB congeners recalcitrant to aerobic degradation and only aerobic microorganisms are capable of mineralizing lesser-chlorinated congeners. As such, determining probes and primers effective for rapid determination and isolation of anaerobic microorganisms that are capable of dechlorination of persistent chlorinated compounds would be advantageous for the bioremediation of a contaminated site.

### SUMMARY OF THE INVENTION

The present invention provides a set of primers for use in a detection assay for detecting PCB dechlorinating organisms in a sample.

In one aspect, the present invention provides a set of primers useful for the identification of new PCB dechlorinating bacteria, wherein the set of primers include both SEQ ID NOs: 1 and 2 and any nucleotides sequences that are complementary to same, have more than 98% identity, or that hybridize under high stringency conditions of 0.1×SSC, 0.1% SDS at 65° C.; and that hybridizes with 16S rRNA of a bacteria.

In another aspect the present invention provides for an isolated bacterial organism identified by using at least one primer selected from SEQ ID NOs: 1, 2 and 3, wherein said bacterial organism has the ability to dechlorinate PCB chlorinated compounds. Preferably, the isolated bacterial organism is a bioremediative microorganism for PCB dechlorination comprising a 16S ribosomal subunit nucleic acid sequence selected from the group consisting of:

(a) a 16S ribosomal subunit nucleic acid sequence consisting of SEQ ID NO: 4;

(b) a nucleic acid sequence that has more than 95% identity to the nucleic acid sequence of SEQ ID NO: 4; and

(c) a nucleic acid sequence fully complementary to the nucleic acid of (a); and

wherein the isolated bioremediative microorganism anaerobically dechlorinates chlorinated biphenyls.

In yet another aspect, the present invention provides a method for identifying a PCB dechlorinating bacterial organism comprising: (i) extracting genomic DNA from a bacteria cell suspected of being able to dechlorinate PCB chlorinated compounds; (ii) probing the extracted genomic DNA with at least one probe having a sequence selected from the group consisting of SEQ ID NOs: 1 and 2, under suitable hybridization conditions, wherein the identification of a hybridizable nucleic acid fragment confirms the presence of a bacteria capable of dechlorinating PCB chlorinated compounds.

Similarly, in another aspect, the present invention provides a method for identifying a PCB dechlorinating bacterial organism comprising (i) extracting genomic DNA from a bacteria cell suspected of being able to dechlorinate PCB chlorinated compounds; and (ii) amplifying the extracted genomic DNA with a primer set comprising at least one sequence as set forth in SEQ ID NO: 1 or SEQ ID NO: 2, and any nucleotides sequences that are complementary to same, have more than 98% identity and/or that hybridize under high stringency conditions of 0.1×SSC, 0.1% SDS at 65° C., such that amplification products are generated wherein the presence of amplification products confirms the presence of a PCB dechlorinating bacterial organism. Preferably both SEQ ID NO: 1 or SEQ ID NO: 2 are included in the primer set.

The invention additionally provides a method for identifying a PCB dechlorinating bacterial organism comprising:
(i) extracting total cellular RNA from a bacteria cell suspected of being able to dechlorinate PCB chlorinated compounds;
(ii) synthesizing complementary DNA strands to the extracted rRNA using a reverse transcriptase and at least one oligonucleotide primer having a sequence selected from the group consisting of SEQ ID NO: 1 and SEQ ID NO: 2;
(iii) amplifying the newly generated complementary DNA strands to the extracted rRNA using at least one oligonucleotide primer corresponding to at least one of the sequences of step (ii) such that amplification products are generated; wherein the presence of amplification products confirms the identification of a PCB dechlorinating bacterial organism.

A still further aspect of the present invention provides a method for the dechlorination of PCB chlorinated compounds comprising:

contacting a PCB chlorinated compound with an isolated bacterial organism consisting of a 16S rDNA sequence as set forth in SEQ ID NO: 4 under conditions suitable for PCB dechlorination to occur.

In yet another aspect, the present invention provides a method for separating sub-families of PCB dechlorinating bacterial organisms comprising:
(i) extracting total cellular rRNA from a bacteria cell suspected of being able to dechlorinate PCB chlorinated compounds;
(ii) synthesizing complementary DNA strands to the extracted rRNA using a reverse transcriptase and at least one oligonucleotide primer selected from SEQ ID NO: 1 or SEQ ID NO: 2;
(iii) amplifying the newly generated complementary DNA strands to the extracted rRNA of step (ii) using at least one oligonucleotide primer selected from SEQ ID NO: 1 or SEQ ID NO: 2 such that amplification products are generated; and
(iv) separating the amplification products by Denaturing Gradient Gel Electrophoresis.

The invention also contemplates methods of determining the bioremediative potential of a chlorinated biphenyl-containing site, comprising:
contacting a nucleic acid molecule, including a nucleic acid sequence selected from the group consisting of:
   (i) a nucleic acid sequence that has more than 98% identity to a nucleic acid sequence selected from the group consisting of SEQ ID NO: 1 and SEQ ID NO: 2;
   (ii) a nucleic acid sequence fully complementary to a nucleic acid of (a),
      with a nucleic acid-containing sample from the biphenyl-containing site under approximately stringent hybridization conditions, and determining positive bioremediative potential in the event that hybridization is detected.

In a further aspect, the invention relates to a method of monitoring a chlorinated biphenyl-containing site, comprising conducting serial observations using methods described herein.

In a still further aspect, the present invention provides an enrichment culture that reductively dechlorinates PCBs wherein the enrichment culture comprises an isolated bacterial organism comprising a 16S ribosomal subunit nucleic acid sequence selected from the group consisting of:
(a) a nucleic acid sequence that has more than 99% identity to a nucleic acid sequence of SEQ ID NO: 4; and
(b) a nucleic acid sequence fully complementary to a nucleic acid of (a); and

wherein the isolated bioremediative microorganism anaerobically dechlorinates chlorinated biphenyls under conditions suitable for PCB dechlorination to occur.

The present invention provides compositions and methods for anaerobically degrading extensively chlorinated congeners to primarily mono- and dichlorobiphenyls, and in one illustrative aspect contemplates the treatment of PCBs with an anaerobic consortium of bacteria, followed by treatment with an aerobic consortium of bacteria, to maximize the overall degradation of PCBs.

The present invention facilitates bioremediation treatment in which dechlorination composition(s) of the invention can be seeded into clean sediments, to provide sedimentary composition(s) comprising the clean sediment material, mixed with nutrients and dechlorinating microorganisms. The sedimentary composition including the active microbial agent can be deposited over PCB-contaminated material at sites containing PCBs, such as marine or riparian sites having native sediments contaminated with PCBs, landfill sites containing PCB waste, etc. Such “capping treatment” approach has major advantages over current PCB contamination removal techniques, such as dredging of river and ocean sites, which are simply relocation measures and do not provide in situ elevation of PCBs at the locus of contamination.

The invention may also be variously embodied to carry out corresponding processes for treatment of water containing PCBs. In such processes, the dechlorinating composition of the invention can be presented in a fixed bed, bioreactor, biofilter, etc., for continuous treatment of PCB-contaminated water by flow thereof through the dedicated treatment system.

A further aspect of the present invention provides a diagnostic nucleic acid gene fusion useful in Denaturing Gradient Gel Electrophoresis (DGGE) having the general structure: SS-GC, wherein:
(i) SS is a signature sequence selected from the group consisting of SEQ ID NO: 1 and SEQ ID NO: 2; and
(ii) GC is a GC clamp sequence having the sequence as set forth in SEQ ID NO: 5.

A still further aspect, of the present invention provides for a selective PCR primer set that amplifies the 16S rRNA genes of a broad range of species within the “dehalogenating Chloroflexi” including Dehalococcoides spp. and the o-17/DF-1 group, wherein the Forward primer Chl348F (5′-GAGGCAGCAGCAAGGAA-3′) (SEQ ID NO: 1) is specific for Chloroflexi and reverse primer Dehal884R (5′-GGCGGGACACTTAAAGCG-3′) (SEQ ID NO: 2) is specific for putative dechlorinating species. For DGGE analyses a GC clamp (CGC CCG CCG CGC GCG G)(SEQ ID NO: 5) was added to primer Chl348F (5′-CGC CCG CCG CGC GCG GGA GGC AGC AGC AAG GAA-3′) (SEQ ID NO: 3)(Genosys Biotechnologies), and designated Chl348FGC.

The invention, as will be appreciated more fully from the ensuing description, provides a fundamental advance in the art of treatment and destruction of PCBs, and may be applied in a wide variety of potential uses and applications for abating of PCBs, as will be appreciated by those skilled in the art.

### BRIEF DESCRIPTION OF THE DRAWINGS

FIG. 1 shows alignment of primer with 16S rRNA genes of o-17, DF-1, DEH10, Dehalococcoides ethenogenes 195 (DHE) and Chloroflexus aurantiacus (Chl.). Numbering is based on the E. coli 16S rDNA positions. Panel on the right shows an agarose gel of PCR products using primers Chl348F and Dehal884R and plasmids with 16S rDNA gene templates from the organisms indicated. The top lane is the DNA size marker.

FIG. 2 shows quantitative results of 16S rRNA genes during active dechlorination of PCBs 132 and 91, showing mol % of parent compound in active culture (□) and sterile control (▪); MPN-PCR analyses of 16S rDNA copies per μl of DNA in active culture (∘) and sterile control (●).

FIG. 3 is a schematic drawing showing development of differential PCR products for quantitation with competitive PCR.

FIG. 4 is a schematic drawing showing the procedure for quantitation with competitive PCR.

FIG. 5 shows quantification of PCB dechlorinating bacterium DF-1 using competitive PCR with selective primers. Numbers agreed with direct counts of DAP1-stained cells under the microscope.

FIG. 6 shows a flow chart showing protocol for detection and quantitation of putative PCB dechlorinating bacteria in sediments with competitive PCR.

FIG. 7 is a chart showing results of analyses of sediments with different levels of PCB contamination. Sites 16 and 18 were PCB free, site 5 had low levels and the remaining sites had varying degrees of higher levels PCB contamination.

FIG. 8 shows the qualitative analyses of 16S rRNA genes during active dechlorination of PCBs 132 and 91. DGGE results from dechlorinating cultures and the no PCB controls. Lanes 1-4 from left are from sediment microcosms with added PCB and lanes 5-7 from left do not contain PCB. All bands were excised and sequenced. Assay shows DEH10 and SF-1 enriched in sediment microcosms actively dechlorinating PCB 132 and PCB 91, respectively. Bands in far right lane are products from (from the top) DEH10 and SF1.

FIG. 9 shows HaeIII and HhaI restriction endonuclease digestions of 24 clones generated with PCR with Chl348F and Dehal884R from an actively dechlorinating sediment microcosm enriched with PCB 101. Result shows enrichment for a single clone in 22 out or 24 lanes.

FIG. 10 shows the phylogenetic analysis (neighbor joining) of 16S rRNA genes from selected members of the dehalogenating Chloroflexi group, including subgroups described by Hendrickson et al. Tree reconstruction was based on 1027 positions except for DEH10 (101), DEH10 (132) and SF1, which were shorter (450 basepairs) and added by ARB parsimony. The tree was rooted with Chloroflexus aggregans (D32255). Bootstrap values over 50 are indicated at the branch points. Scale bar indicates 10 substitutions per 100 nucleotide positions. Primers Chl348FGC/Dehal884R and Chl348F/Dehal884R detect all species within the bracket labeled “dehalogenating Chloroflex,” wherein “dehalococcoides” primers only detect species within the Dehalococcoides group.

### DETAILED DESCRIPTION AND PREFERRED EMBODIMENTS OF THE INVENTION

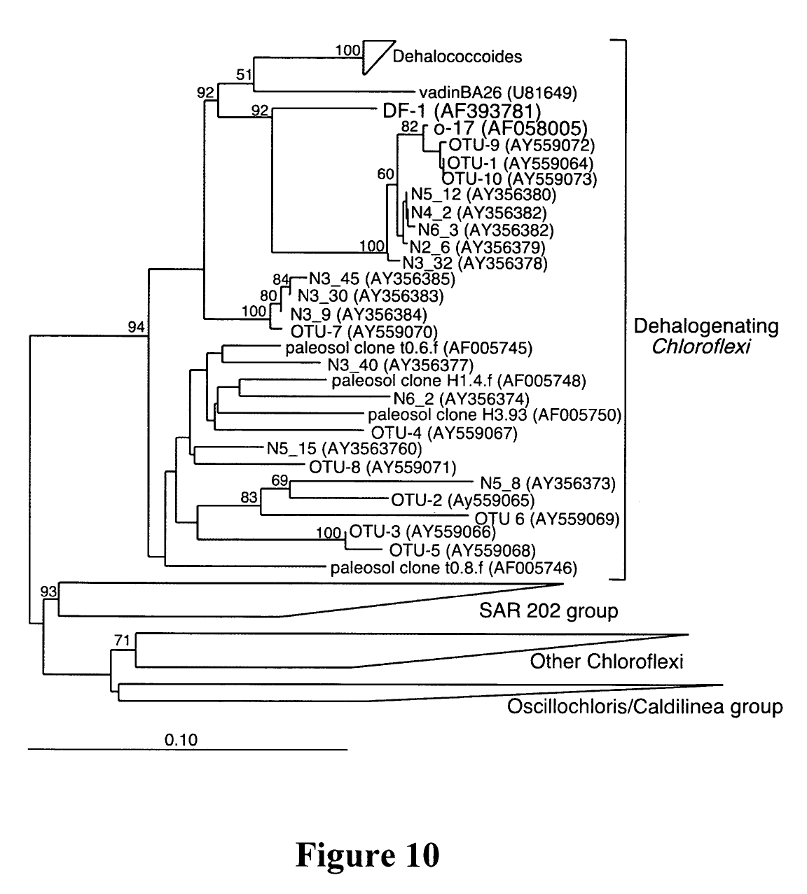

Sequence similarity among Dehalococcoides strains is very high (>98%), while the similarity between the o-17, DF-1 group and the Dehalococcoides strains is less than 90%. Nevertheless, all these microorganisms form a monophyletic clade within the Chloroflexi as shown in FIG. 10 and this group appears to have the ability to use various halogenated compounds as electron acceptors. The present invention includes the development of new PCR primers for the 16S rRNA genes of members of the dehalogenating Chloroflexi group that includes both Dehalococcoides spp and o-17/DF-1-like microorganisms. Using these comprehensive primers in a qualitative DGGE assay provides for the identification of dechlorinating microorganisms in actively dechlorinating cultures. Results set forth herein show that two phylotypes, one closely related to o-1 7/DF-1 and the second a Dehalococcoides sp., sequentially dechlorinate the double flanked and single flanked meta chlorines of PCB 132 in Baltimore Harbor sediment microcosms.

The term “dechlorinating bacteria” refers to any bacterial species or organism that has the ability to remove at least one chloride atom from a chlorinated organic compound. Dechlorinating bacteria may have the ability to grow on chlorinated organics as a sole electron acceptor, or may prefer degradation using an alternate energy source.

The term “oligonucleotide” refers to primers, probes, oligomer fragments to be detected, labeled-replication blocking probes, and oligomer controls, and shall be generic to polydeoxyribonucleotides (containing 2-deoxy-D-ribose), to polyribonucleotides (containing D-ribose) and to any polynucleotide which is an N-glycoside of a purine or pyrimidine base (nucleotide), or modified purine or pyrimidine base. Also included in the definition of “oligonucleotide” are nucleic acid analogs (e.g., peptide nucleic acids) and those that have been structurally modified (e.g., phosphorothioate linkages). There is no intended distinction between the length of a “nucleic acid,” “polynucleotide” or an “oligonucleotide.”

The term “primer” refers to an oligonucleotide (synthetic or occurring naturally), which is capable of acting as a point of initiation of nucleic acid synthesis or replication along a complementary strand when placed under conditions in which synthesis of a complementary stand is catalyzed by a polymerase.

The term “probe” refers to an oligonucleotide (synthetic or occurring naturally), which is significantly complementary to a “fragment” and forms a duplexed structure by hybridization with at least one strand of the fragment.

The term “complementary” is used to describe the relationship between nucleotide bases that are hybridizable to one another. For example, with respect to DNA, adenosine is complementary to thymine and cytosine is complementary to guanine.

A nucleic acid molecule is “hybridizable” to another nucleic acid molecule, such as a cDNA, genomic DNA, or RNA, when a single-stranded form of the nucleic acid molecule can anneal to another single-stranded nucleic acid molecule under the appropriate conditions of temperature and solution ionic strength to form a double-stranded nucleic acid. Hybridization and washing conditions are well known to those skilled in the art. The conditions of temperature and ionic strength determine the “stringency” of the hybridization. For preliminary screening for homologous nucleic acids, low stringency hybridization conditions, corresponding to a Tm of 55° C., can be used, e.g., 5×SSC, 0.1% SDS, 0.25% milk, and no formamide; or 30% formamide, 5×SSC, 0.5% SDS. Moderate stringency hybridization conditions correspond to a higher Tm, e.g., 40% formamide, with 5× or 6×SSC.

The term “amplification product” refers to portions of nucleic acid fragments that are produced during a primer directed amplification reaction. A typical method of primer directed amplification includes polymerase chain reaction (PCR). In PCR, the replication composition would include for example, nucleotide triphosphates, two primers with appropriate sequences, DNA or RNA polymerase and proteins. These reagents and details describing procedures for their use in amplifying nucleic acids are provided in U.S. Pat. No. 4,683,202 (1987, Mullis, et al.) and U.S. Pat. No. 4,683,195 (1986, Mullis, et al.), the contents of which are hereby incorporated by reference herein.

The term “reverse transcription followed by polymerase chain reaction”, or “RT-PCR”, refers to a sensitive technique for quantitative analysis of gene expression, cloning, cDNA library construction, probe synthesis, and signal amplification in situ hybridizations. The technique consists of two parts: synthesis of cDNA from RNA by reverse transcription (RT), and amplification of a specific cDNA by polymerase chain reaction (PCR). Reverse Transcriptase is an RNA dependent DNA polymerase that catalyses the polymerization of nucleotides using template DNA, RNA or RNA: DNA hybrids. It is important to utilize a total RNA isolation technique that yields RNA lacking significant amounts of genomic DNA contamination, since the subsequent PCR cannot discriminate between cDNA targets synthesized by reverse transcription and genomic DNA contamination.

The term “sequence analysis software” refers to any computer algorithm or software program that is useful for the analysis of nucleotide or amino acid sequences. Typical sequence analysis software will include but is not limited to the GCG suite of programs (Wisconsin Package Version 9.0, Genetics Computer Group (GCG), Madison, Wis.), BLASTP, BLASTN, BLASTX (Altschul et al., J. Mol. Biol. 215:403-410 (1990), and DNASTAR (DNASTAR, Inc., 1228 S. Park St. Madison, Wis. 53715 USA).

In order to enrich the cultured soil samples for PCB dechlorinating bacteria, the samples are contacted with a chlorinated organic compound. A number of chlorinated compounds are suitable for this purpose including, but not limited to: carbontetrachloride, tetrachloroethene, chloroform, dichloromethane, trichloroethene, dichloroethylene, vinyl chloride, and chloroaromatics, dichloropropane, and chlorinated ethane where chlorinated ethenes are preferred. Incubation proceeded for about six months, and cultures were analyzed periodically for the disappearance of the chlorinated organic and the appearance of degradation products. Cultures demonstrating the ability to degrade chlorinated organics were selected for further analysis.

Bacteria from dechlorinating cultures are removed by standard methods and total chromosomal DNA isolated from the microorganisms through a bead mill homogenization procedure. A fragment of the 16S rRNA gene is amplified from the genomic DNA extract by PCR using 16S rDNA primers SEQ ID NOs: 1 and 2 specific for PCB dechlorinating microbes. The 16S rDNA PCR product is cloned and sequenced to confirm its identity according to methods known to those skilled in the art.

In a preferred embodiment the present sequences (SEQ ID NOs: 1 and 2) may be used as primers or to generate primers that may be used in primer directed nucleic acid amplification to detect the presence of PCB dechlorinating bacteria. A variety of primer directed nucleic acid amplification methods are known in the art including thermal cycling methods such as polymerase chain reaction (PCR) and ligase chain reaction (LCR) as well as isothermal methods and strand displacement amplification (SDA). The preferred method is PCR. Typically, in PCR-type amplification techniques, the primers have different sequences and are not complementary to each other. Depending on the desired test conditions, the sequences of the primers should be designed to provide for both efficient and faithful replication of the target nucleic acid.

If a nucleic acid target is to be exponentially amplified, then two primers are used each having regions complementary to only one of the stands in the target. After heat denaturation, the single-stranded target fragments bind to the respective primers that are present in excess.

Following amplification and prior to sequencing, the amplified nucleotide sequence may be ligated to a suitable vector followed by transformation of a suitable host organism with said vector. One thereby ensures a more readily available supply of the amplified sequence. Alternatively, following amplification, the amplified sequence or a portion thereof may be chemically synthesized for use as a nucleotide probe. In either situation the DNA sequence of the variable region is established using methods such as the dideoxy method (Sanger, F. et al. Proc. Natl. Acad. Sci (1977) 74, 5463-5467). The sequence obtained is used to guide the choice of the probe for the organism and the most appropriate sequence(s) is/are selected.

A variety of PCR detection methods are known in the art including standard non-denaturing gel electrophoresis (e.g., acrylamide or agarose), denaturing gradient gel electrophoresis, and temperature gradient gel electrophoresis. Standard non-denaturing gel electrophoresis is the simplest and quickest method of PCR detection, but may not be suitable for all applications.

Denaturing Gradient Gel Electrophoresis (DGGE) is a separation method that detects differences in the denaturing behavior of small DNA fragments (200-700 bp). The principle of the separation is based on both fragment length and nucleotide sequence. In fragments that are the same length, a difference as little as one base pair can be detected. DGGE is primarily used to separate DNA fragments of the same size based on their denaturing profiles and sequence. Using DGGE, two strands of a DNA molecule separate, or melt, when heat or a chemical denaturant is applied. The denaturation of a DNA duplex is influenced by two factors: 1) the hydrogen bonds formed between complimentary base pairs (since GC rich regions melt at higher denaturing conditions than regions that are AT rich); and 2) the attraction between neighboring bases of the same strand, or “stacking”. Consequently, a DNA molecule may have several melting domains with each of their individual characteristic denaturing conditions determined by their nucleotide sequence. DGGE exploits the fact that otherwise identical DNA molecules having the same length and DNA sequence, with the exception of only one nucleotide within a specific denaturing domain, will denature at different temperatures or Tm. When the double-stranded (ds) DNA fragment is electrophoresed through a gradient of increasing chemical denaturant it begins to denature and undergoes both a conformational and mobility change. The dsDNA fragment will travel faster than a denatured single-stranded (ss) DNA fragment, since the branched structure of the single-stranded moiety of the molecule becomes entangled in the gel matrix. As the denaturing environment increases, the ds DNA fragment will completely dissociate and mobility of the molecule through the gel is retarded at the denaturant concentration at which the particular low denaturing domains of the DNA strand dissociate. In practice, the electrophoresis is conducted at a constant temperature (around 60° C.) and chemical denaturants are used at concentrations that will result in 100% of the DNA molecules being denatured (i.e., 40% formamide and 7M urea). This variable denaturing gradient is created using a gradient maker, such that the composition of each DGGE gel gradually changes from 0% denaturant up to 100% denaturant. Of course, gradients containing a reduced range of denaturant (e.g., 35% to 60%) may also be poured for increased separation of DNA.

As used in the present invention the diagnostic nucleic acid gene fusions were made comprising a signature sequence and a GC clamp sequence designed to alter the mobility of the fusion in the gel media. Preferred in the present invention are signature sequences having the SEQ ID NOs: 1 or 2. Preferred GC clamp sequences are those having sequence similarity to the sequence as set forth in SEQ ID NO: 5. The skilled artisan will appreciate that placement of the GC clamp on the sequence is a matter of discretion for the investigator and that the GC clamp sequence may be attached at either 5′ end of the signature sequence.

A suitable method for separating sub-families of PCB dechlorinating bacterial organisms according to the present invention may comprise steps including: (i) extracting total cellular RNA from a bacteria cell suspected of being able to dechlorinate PCB compounds; (ii) synthesizing complementary DNA strands to the extracted rRNA using a reverse transcriptase and at least one oligonucleotide primer corresponding to a portion of a suitable diagnostic gene fusion of the invention such that amplification products are generated; (iii) amplifying the newly generated complementary DNA strands to the extracted rRNA of step (ii) using at least one oligonucleotide primer corresponding to a portion of a suitable diagnostic gene fusion of the invention such that amplification products are generated; and (iv) separating the amplification products by Denaturing Gradient Gel Electrophoresis.

The invention enables the effective dechlorination of chlorinated biphenyls, involving the provision in the chlorinated biphenyl-containing environment of growth conditions for the microorganism(s) such that chlorine is at least partially removed from the chlorinated biphenyl. The microbial dechlorination process is advantageously carried out in the presence of appropriate additives necessary or desirable for dechlorinative action on PCBs being treated. Non-limiting examples of additives that may be used in the practice of the invention include hydrogen, acetate, formate and/or fumarate, as for example may be added to the chlorinated biphenyl-containing environment being treated. For example, dechlorination may be carried out under sufficient conditions to effect dechlorination of flanked chlorine of poly-chlorinated biphenyl present in the environment being treated by the microbial species, utilizing appropriate additives/growth conditions.

The invention contemplates treatment of a PCB-containing environment by inoculation or other introduction of microbially effective agents of the invention to the environment. For example, dechlorinating bacteria in accordance with the invention may be dispersed on a landfill site under appropriate conditions for effect biodegradative action on PCBs in the environment. Such dispersant may include the microbial agent in a nutrient medium, particularly if the PCB-containing environment is nutrient-deficient for such microbial species. The level of biodegradation of the PCBs can be monitored continuously or intermittently to determine the effectiveness of the microbial treatment.

The dechlorination/bioremediation processes of the present invention may if desired be advantageously combined with other bioremediation and waste-degradation methods conventionally employed in the art, to achieve an enhanced decontamination or purification result. The compositions and methods of the invention may be employed for anaerobically degrading extensively chlorinated congeners to primarily mono- and dichlorobiphenyls, e.g., involving the treatment of PCBs with an anaerobic consortium of bacteria in accordance with the invention, followed by treatment with an aerobic consortium of bacteria, to maximize the overall degradation of PCBs.

The invention therefore contemplates the treatment of highly chlorinated PCBs by an anaerobic consortium of microbial species (species that are anaerobically effective for dechlorination of the highly chlorinated congeners), followed by treatment of the correspondingly anaerobically degraded PCBs with an aerobic consortium of microbial species (that are aerobically effective for dechlorination of the partially degraded PCBs).

Such treatment may for example be conducted at a PCB-containing site, e.g., including water, soil and/or sediment, or otherwise with respect to a separated or recovered PCB-containing material or isolated PCBs, in which one or more PCB-degrading anaerobic microorganisms is brought into degradative relationship with the PCB(s) to effect at least partial dechlorination of the PCB(s) under conditions effective for such microbial action. The dechlorinating action may include removal of chlorine substituents from the ortho position of a ring of the PCB, and/or removal of chlorine substituents that are double-flanked by other chloro substituents on the biphenyl ring structure. The microbial consortia employed for such purpose may further contain or be followed in the treatment flow sequence by organisms specifically adapted for dechlorination of para- and meta-chloro substituents, to provide a comprehensive dechlorination treatment of the PCB(s).

The features and advantages of the invention are more fully apparent from the following illustrative examples, which are not intended in any way to be limitingly construed, as regards the invention hereinafter claimed.

### EXAMPLES

Most PCB dechlorinating bacteria have been detected in a diverse group of microorganisms within a deep branch of the Green Non-sulfur bacteria that have been largely undetected in the past. Most of the PCB dechlorinating bacteria are related to the Dehalococcoides spp., which are capable of dehalogenating chlorinated ethenes. One species, Dehalococcoides ethenogenes, completely dehalogenates perchloroethene (PCE) and trichloroethene (TCE) to ethane and commercial PCR primers can detect these microorganisms in environments that generate ethene in PCE and TCE contaminated sites. However, the primers often do not detect microorganisms in sites that do not completely dehalogenate PCE and TCE to ethane. The PCB dechlorinating microorganisms DF-1 and o-17, which are only 89% similar to Dehalococcoides dehalogenate selected PCB congeners, PCE and TCE to only DCE and dehalogenate hexa-, penta-, and tri-chlorobenzene. In light of the fact that the presently described primers Chl348FGC/Dehal884R or Chl348F/Dehal884R can detect Dehalococcoides and species related to Dehalococcoides such as DF-1 and o-17, it is believed that they are also effective for detecting PCE and TCE dehalogenating bacteria at all sites including those that only dechlorinate to DCE.

For concurrent detection of all known dehalogenating microbes within the Chloroflexi, including both Dehalococcoides spp. and non-Dehalococcoides species, a new comprehensive primer set was developed. Forward primer Chl348F (5′-GAGGCAGCAGCAAGGAA-3′) is specific for Chloroflexi and reverse primer Dehal884R (5′-GGCGGGACACTTAAAGCG-3′) is specific for putative dechlorinating species. For DGGE analyses a GC clamp (30) was added to primer Chl348F (5′-CGC CCG CCG CGC GCG GGA GGC AGC AGC AAG GAA-3′) (Genosys Biotechnologies), and designated Chl348FGC. The primers were checked for compatibility and possible self-annealing using Primer Express (Applied Biosystems, Foster City, Calif.).

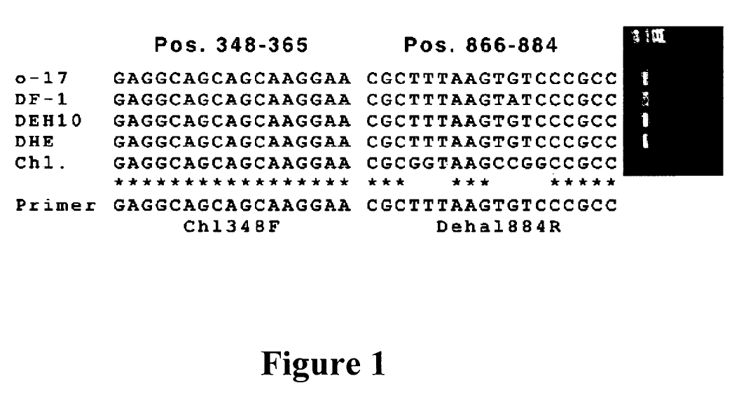

FIG. 1 shows the alignment of 16S rRNA genes of o-17, DF-1, DEH10, Dehalococcoides ethenogenes 195 (DHE) and Chloroflexus aurantiacus (Chl.). Numbering is based on the E. coli 16S rDNA positions. Panel on the right shows an agarose gel of PCR products using primers Chl348F and Dehal884R and plasmids with 16S rDNA gene templates from the organisms indicated. The top lane is the DNA size marker. The detection limit of these primers was ≧10⁵ copies per 50 μl PCR reaction mixture with 26 PCR cycles and 8 μl loaded in agarose gel. The detection limit in 8 μl with 40 PCR cycles ranged between 10 and 65 gene copies per 50 μl PCR reaction mixture for o-17, DF-1 and Dehalococcoides sp. DEH10. The addition of up to 10 μg Chloroflexus aurantiacus DNA, which is related to the dehalogenating species but not detected, had no effect on the sensitivity of the assay.

The sets of primers described herein are used for both quantitative and qualitative analysis to selectively detect PCB dechlorinating and related bacteria. Quantitative analysis is used for PCR methods including both Most Probable Number (MPN)PCR and Competitive PCR.

Dehalogenating Chloroflexi are enumerated by MPN-PCR using primers Chl348F and Dehal884R. Extracted DNA samples (10 μg/mL) are serially diluted 10-fold and amplified using the GeneAmp PCR kit (PE Applied Biosystems, Foster City, Calif.). The reaction contained 1×PCR buffer, a mixture of dNTPs (200 nM each), 1.5 mM MgCl₂, 160 nM of each primer, 192 mM dimethylsulfoxide (DMSO) and 1 unit AmpliTaq DNA polymerase in 50 μl reactions. Amplification was performed in a PTC200 thermal cycler (MJ Research, Watertown, Mass.) with the following cycle parameters: an initial 1 min denaturing step of at 95° C., followed by 40 cycles of denaturation for 45 s at 95° C., annealing for 45 s at 60° C., elongation for 45 s at 72° C., with a final 30 min extension step at 72° to reduce the occurrence of artificial double bands (25). PCR products were checked for correct size and yield on a 0.8% (wt/vol) TAE agarose gel (Fisher Biotech, NJ.). Dehalogenating Chloroflexi 16S rRNA gene copies per μl of DNA sample was determined using a standard Most Probable Numbers table (9). Dilutions of a plasmid with the 16S rRNA gene of the PCB dechlorinating microorganisms o-17 (14), DF-1 (45) and Dehalococcoides sp. DEH10 were used as controls and to determine the sensitivity of the assay. In order to test whether non-homologous DNA would interfere with the MPN assay, 10 ng DNA from a Chloroflexus aurantiacus isolate were added to dilution series and MPN numbers calculated as described above.

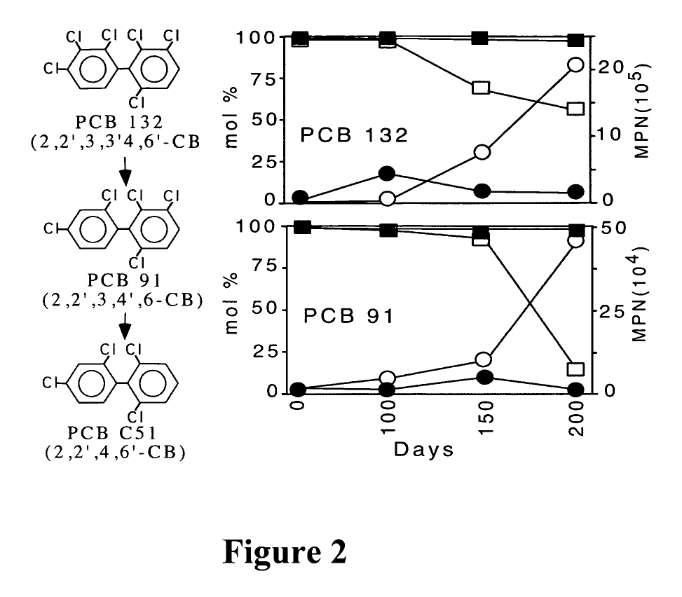

The MPN data set forth in FIG. 2 shows the number of 16S rRNA genes during active dechlorination of PCBs 132 and 91 (mol % of parent compound in active culture (□) and sterile control (▪); MPN-PCR analyses of 16S rDNA copies per μl of DNA in active culture (∘) and sterile control (●)). It is apparent that during active dechlorination of PCB 132 and PCB 91 the cultures exhibited a 20-fold and 50-fold increase in dehalogenating Chloroflexi 16S rRNA gene copies, respectively. All microcosms incubated with PCB 132 and 91 exhibited reductive dechlorination in the meta position. PCB 132 was dechlorinated to PCB 91 in a meta position flanked by two chlorines. PCB 91 was then dechlorinated in a meta position flanked by one chlorine. It was found that the organism represented by the 16S rRNA gene clone DEH10 was responsible for both double flanked meta dechlorination of PCB132.

The apparent preference for double and then single flanked chlorines could be explained based on the chemistry of chlorinated biphenyls. It has been proposed that the first step in microbial reductive dechlorination is the transfer of an electron to the chlorinated biphenyl and the formation of a carbanion intermediate (31). The negative charge will be stabilized through resonance throughout the biphenyl molecule. The ability of the molecule to be stabilized through resonance will also influence the overall reactivity, or standard potential (E°), of different PCB congeners. Generally, higher chlorinated congeners have higher E° values, and are more reactive in environments with low redox potential. Furthermore, PCB molecules with ortho chlorines are less planar, have lower E° values, and are chemically less reactive (11, 34). It is important to keep in mind that these differences in reactivity are solely based on chemical properties, and that the interaction of chlorinated biphenyls with reductive dehalogenases may change the reactive potential of PCB congeners. Nevertheless, Huang et al. (23) indicates that there is a correlation between E° values in microemulsions and ease of reduction by anaerobic bacteria.

Competitive PCR using Chl348F/Dehal884R Primers

Competitive RT-PCR precisely quantitates a message by comparing RT-PCR product signal intensity to a concentration curve generated by a synthetic competitor sequence. The competitor transcript is designed for amplification by using the same primers and with the same efficiency as the endogenous target. However, the competitor produces a different-sized product so that it can be distinguished from the endogenous target product by gel analysis. The competitor is carefully quantitated and titrated into replicate RNA samples. Standard control experiments are used to find the range of competitor concentration where the experimental signal is most similar. Finally, the mass of product in the experimental samples is compared to the curve to determine the amount of a specific RNA present in the sample. Thus, initially, a DNA competitor must be constructed from the primer set Chl348F/Dehal884R.

The Competitive DNA construction kit (#RR017) from TaKaRa Bio Inc, Japan was used for construction of the DNA competitor. The primer set for the dechlorinating chloroflexi consists of the following primers:

Forward:
Chl348. [5′ -GAG GCA GCA GCA AGG AA-3′]
Reverse:
Dehal884. [5′ - GGC GGG ACA CTT AAA GCG-3′]

The preparation of DNA competitor follows the instructions of the TaKaRa DNA Construction Kit (#RR017). The present primers are being used for PCR amplification of a given target, and as such two additional primers are used to generate a construct for competitive RT-PCR. The additional primers are constructed around the original primers so that binding at the primer binding site of the competitor represents that of the endogenous target. However, there is a different in size so that to produce PCR products that are approximately 20% larger or smaller than the endogenous target to allow effective separation and analysis by gel electrophoresis.

Primer Sequences for Amplification of Target DNA:

Sense primer (A):
Chl348. [5′ -GAG GCA GCA GCA AGG AA-3′]
Sense primer (B):
Dehal884. [5′ - GGC GGG ACA CTT AAA GCG-3′]

Determined the amplified region of the template and design the primers (C) and (D).

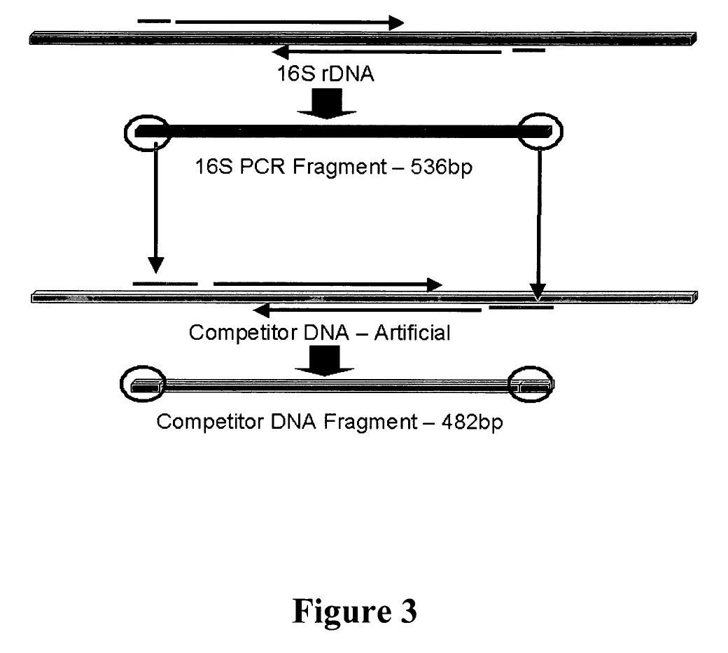

The difference in size between the amplified target DNA and DNA competitor was designed within 20%. The size of the amplified target is 536 bp as shown in FIG. 3. The total length of primer (A) and primer (B) is 35 bp. Since the difference in size between target and competitor should be less than 20%, both the Comp400R and CompF primers were used.

Sense sequence for primer (C):
CompF [5′-GTACGGTCATCATCTGACAC-3′]
Sense sequence for primer (D):
Comp400R [5′GCGTGAGTATTACGAAGGTG-3′]

The sequences for DNA competitor preparation are determined by the sequences (A), (B), (C) and (D).

Sense sequence for primer (A + C) =
(E): Ch2-348F + CompF
[5′- GAGGCAGCAGCAAGGAA—GTACGGTCATCATCTGACAC-3′]
Sense sequence for primer (B + D) =
(F): Dehal-884R + Comp400R
[5′- GGCGGGACACTTAAAGCGGCGTGAGTATTACGAAGGTG-3′]

Extracted DNA from the following cultures and plasmids were used:
DF-1 (dechlorinating organism)
O-17 (dechlorinating organism)
DH10 (dechlorinating organism)
SF (dechlorinating organism)
Desulfovibrio (non dechlorinating organism)
E. Coli (non dechlorinating organism)
   Samples Containing Dechlorinating Cultures:

Enrichment Cultures of DF-1 in Co-Culture with Desulfovibrio

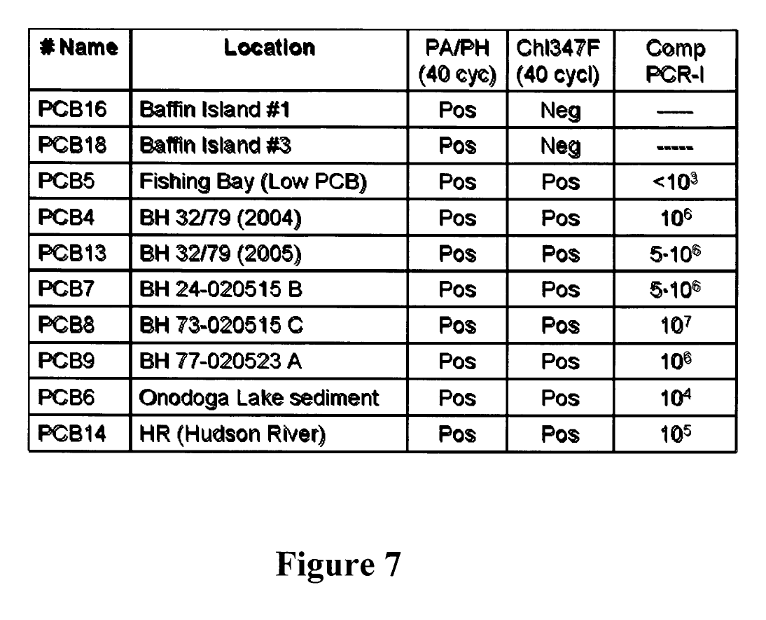

Sediment samples containing unknown dechlorinating bacteria as defined in FIG. 7, including PCB16, PCB18, PCB5, PCB4, PCB13, PCB7, PCB8, PCB9, PCB6 and PCB14.

Competitor DNA was prepared by combining the following components.
2× Premix solution (25 μl)
Sense primer (E) (20 pmol/μl) (0.5 μl)
Antisense primer (F) (20 pmol/μl) (0.5 μl)
dH₂O (24 μl)
Total 50 μl
   PCR program
94° C. 30 sec
60° C. 30 sec
72° C. 30-60 sec
Cycles: 30
   Competitor DNA was Purified as Follows:

Use SUPREC™-02 to purify the DNA competitor (remove excess primers, dNTPs).
1. Transfer PCR product to a fresh tube.
2. Add TE buffer to make a total volume of 400 μl (10 mM Tris-HCl, 1 mM EDTA, pH 8.0).
3. Transfer the solution to the ultrafiltration cassette portion of SUPREC-02
4. Centrifuge at 1500 G (4000 rpm) for 8 minutes.
5. Discard filtrate. Add TE buffer to the solution remaining in the ultrafiltration cassette (up to 400 μl).
6. Centrifuge at 1500 G (4000 rpm) for 8 minutes.
7. Repeat step 5+6 until the DNA solution reaches the desired volume (50 μl).
8. Analyze a small portion of the DNA solution by electrophoresis on a 1.5% agarose gel.
   Plasmid Copy Numbers were Calculated as Follows:

Measure the OD (260 nm) of the purified competitor.
Copies/μl=(OD₂₆₀×50 (ng/μl)×10⁻⁹×6×10²³)/(bp×660). Bp=482
Copies/μl=(OD₂₆₀×9,430¹²) (For this specific DNA Competitor)

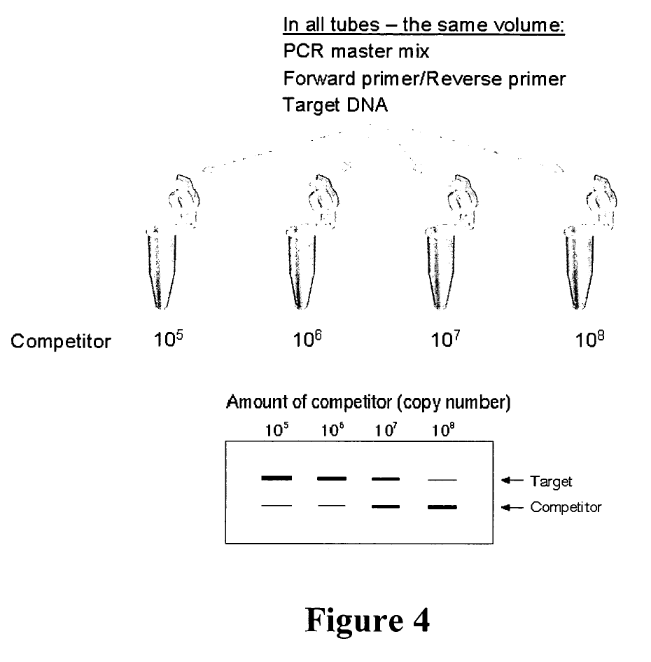

FIG. 4 shows the procedure for quantitation with competitive PCR. In competitive PCR, standard and target sequences compete for the same primer sequences and so amplification takes place in a competitive fashion. A fixed (unknown) quantity of target DNA is amplified with a dilution series of competitor DNA. As the concentration of competitor added to each tube at the start of the reaction is precisely known, the initial concentration of target cDNA in the sample can be readily calculated. Target and competitor PCR products can be most simply distinguished by designing the competitor to have a slight size difference (<20%) that should not alter reaction efficiency. The products are separated by agarose gel electrophoresis and quantified.

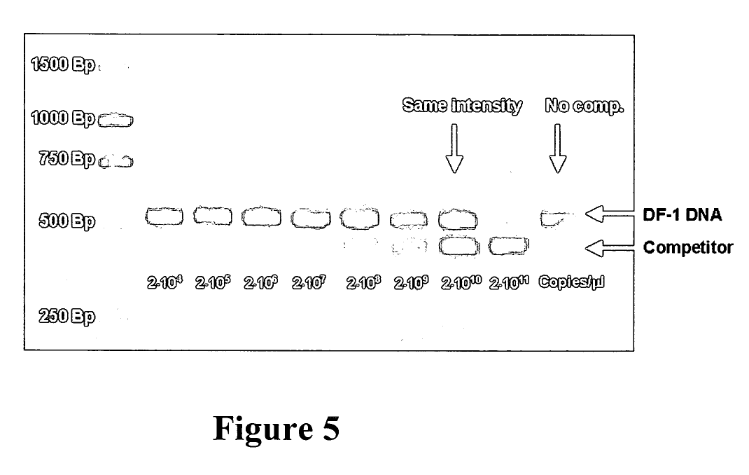

FIG. 5 shows the results of quantification of PCB dechlorinating bacterium DF-1 using competitive PCR with selective primers. Once amplification of target DNA is performed with coexistence of DNA competitor, competitive PCR occurs due to the competition for the use of the primers. Because of the competition, the ratio of the amount between two amplified products reflects the ratio between the target DNA and DNA competitor. So, the amount of the target DNA can be estimated by comparing with the concentration of DNA competitor. The number of copies, (2×10¹⁰ for the target sequence) agreed with direct counts of DAPI-stained cells under the microscope.

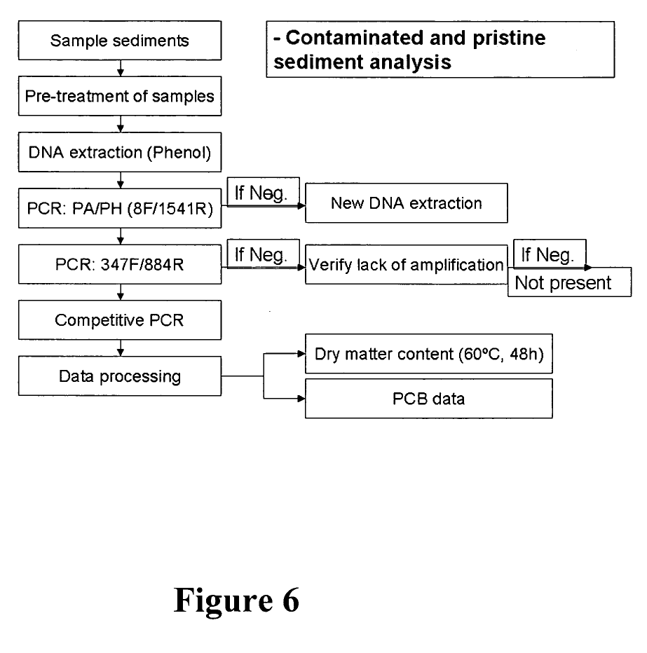

FIG. 6 provides a flow chart setting forth the process steps for detection and quantitation of putative PCB dechlorinating bacteria in sediments with competitive PCR. The results of competitive PCR for the samples from the different testing sites are shown in FIG. 7. Sites 16 and 18 were PCB free, site 5 had low levels and the remaining sites had varying degrees of higher levels PCB contamination.

Qualitative analyses of PCB dechlorinating bacteria and related species with Chl348F/Dehal884R using Denaturing Gradient Gel Electrophoresis (DGGE).

For DGGE analyses a GC clamp(30) was added to primer Chl348F (5′-CGC CCG CCG CGC GCG GGA GGC AGC AGC AAG GAA-3′) (Genosys Biotechnologies), and designated Chl348FGC (SEQ ID NO 3). PCR reactions with 10 ng DNA were performed using the GeneAmp PCR kit (PE Applied Biosystems, Foster City, Calif.). The reaction contained 1×PCR buffer, a mixture of dNTPs (200 nM each), 1.5 mM MgCl₂, 160 nM of each primer, 192 mM dimethylsulfoxide (DMSO) and 1 unit AmpliTaq DNA polymerase in 50 μl reactions. Amplification was performed in a PTC200 thermal cycler (MJ Research, Watertown, Mass.) with the following cycle parameters: an initial 1 min denaturing step of at 95° C., followed by 26 cycles of denaturation for 45 s at 95° C., annealing for 45 s at 60° C., elongation for 45 s at 72° C., with a final 30 min extension step at 72° to reduce the occurrence of artificial double bands (25). The sensitivity of the DGGE assay with the PCR conditions described above was determined by dilution of plasmids containing the 16S rRNA gene of o-17 (14). PCR products were checked for correct size and yield on a 0.8% (wt/vol) TAE agarose gel (Fisher Biotech, N.J.). 26 PCR cycles were used in the DGGE analysis to decrease the PCR bias. DGGE was performed as described by Watts et al. (43) using the D-Code Universal Mutation Detection System (Bio-Rad, Hercules, Calif.). The 6% (wt/vol) polyacrylamide gels (Sigma, St. Louis, Mo.) contained a 39-48% denaturing gradient and fragments were separated by electrophoresis for 18 hours at 75 V. The gels were stained with SYBR-Green 1 DNA stain (Molecular Probes, Eugene, Oreg.) and visualized using a Storm Phosphorlmager (GE Healthcare, Piscataway, N.J.). DGGE bands of interest were excised and eluted from the polyacrylamide gel by incubation in 30 μl TE overnight at 4° C. PCR and DGGE were repeated twice to assure purity of each eluted band and the last PCR reaction used primers without the GC clamp before DNA sequencing by standard methods.

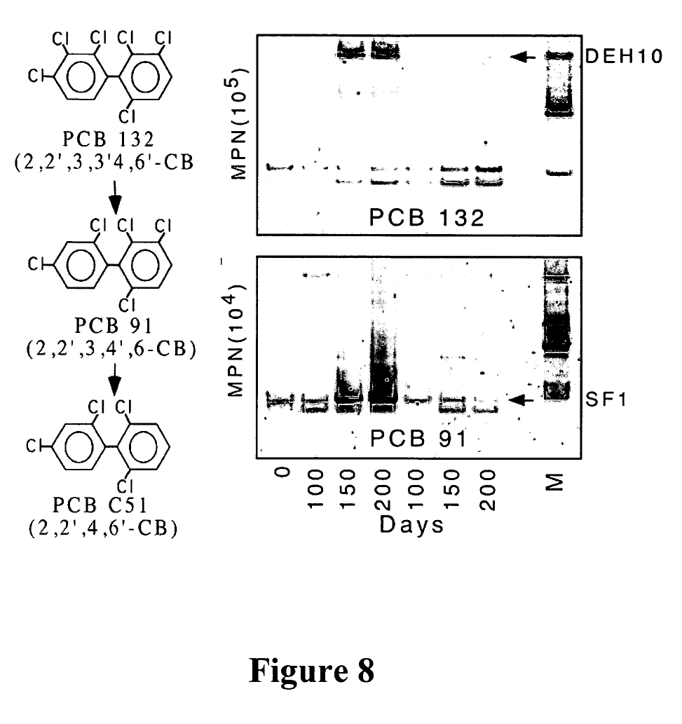

FIG. 8 shows the analyses of 16S rRNA genes during active dechlorination of PCBs 132 and 91. DGGE results from dechlorinating cultures and the no PCB controls. Lanes 1-4 from left are from sediment microcosms with added PCB and lanes 5-7 from left do not contain PCB. All bands were excised and sequenced. The assay shows that DEH10 and DF-1 enriched in sediment microcosms actively dechlorinated PCB 132 and PCB 91, respectively. Bands in far right lane are products from (from the top) DEH10 and SF1. Phylotype SF1 was clearly enriched in the microcosm dechlorinating PCB 91 compared to the no-PCB control. Phylotype SF1 is more closely related to o-17 and DF-1, but most similar to environmental clones from Baltimore Harbor retrieved using primers 14F and Dehal1265R (41, 42). Although the DNA concentrations were normalized among samples and PCR cycles were kept at a minimum to minimize PCR biases, DGGE is a semi-quantitative method. The use of MPN-PCR confirmed the results observed using the DGGE assay. Dehalococcoides sp. DEH10 and phylotype SF1 use the different PCB congeners for dehalorespiratoration. Increases in 16S rRNA gene copies were only observed in cultures actively dechlorinating PCBs, which suggests that PCB dechlorinating activity is linked to growth of both Dehalococcoides sp. DEH10 and bacterium SF1.

Amplified Ribosomal DNA Restriction Analysis (ARDRA) PCR:

For ARDRA, PCR fragments were generated by PCR with Chl348F and Dehal884R. Plasmid libraries were generated in pCR2.1 vector (Invitrogen, Carlsbad, Calif.) according to manufacturer's instructions and screened by restriction analysis after digestion with the endonucleases HaeIII and HhaI (32). DNA restriction fragments were separated by gel electrophoresis on a 3% Trevigel at 25V for 3 hours at 0° C.

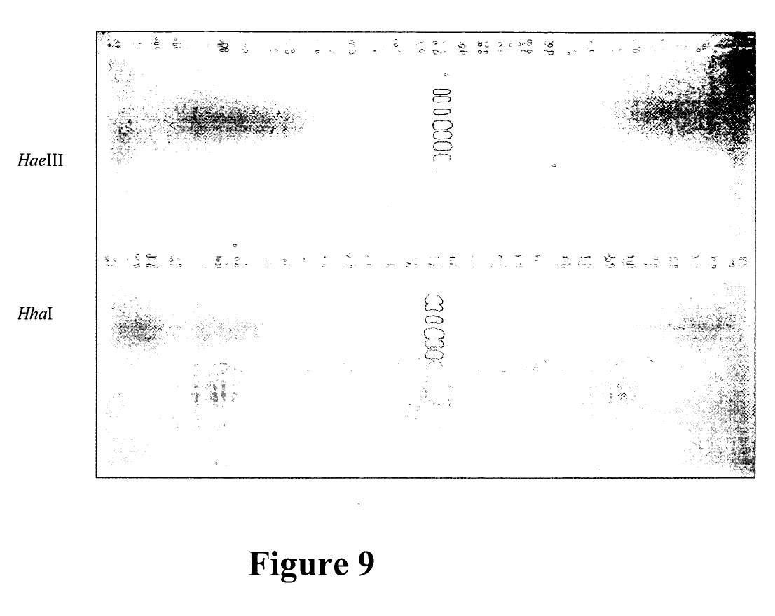

FIG. 9 shows HaeIII and HhaI restriction endonuclease digests of 24 clones generated with PCR with Chl348F and Dehal884R from an actively dechlorinating sediment microcosm enriched with PCB 101. Result shows enrichment for a single clone in 22 out or 24 lanes.

Isolation of PCB dechlorinating microorganisms has proven difficult (6, 13, 14, 32, 43-45). Several isolates in the Dehalococcoides group have been reported (2, 12, 15, 21, 27), but a direct link between growth and PCB dechlorination activity has not heretofore been shown for any of these isolates. The development of primers targeting a broader range of dehalogenating Chloroflexi as in the present primers, including not yet cultured microorganisms, is an important advance in the study of this diverse group of Bacteria and is necessary for their in situ quantitation. The results set forth herein confirm that individual species within the dechlorinating Chloroflexi exhibit a limited range of congener specificity. Importantly, the comprehensive primer set developed herein amplifies both groups of PCB dechlorinating bacteria within the “dehalogenating Chloroflexi” lade in a single PCR reaction.

### REFERENCES

The contents of the following references are hereby incorporated by reference herein for all purposes.
1. Adrian, L., and H. Görisch. 2002. Microbial transformation of chlorinated benzenes under anaerobic conditions. Res. Microbiol. 153:131-137.
2. Adrian, L., U. Szewzyk, J. Wecke, and J. Görisch. 2000. Bacterial dehalorespiration with chlorinated benzenes. Nature 408:580-583.
3. Alder, A. C., M. M. Haggblom, S. R. Oppenheimer, and L. Y. Young. 1993. Reductive dechlorination of polychlorinated biphenyls in anaerobic sediments. Environ. Sci. Technol. 27:530-538.
4. Baker, J., R. Mason, J. Cornwell, J. Ashley, J. Halka, and J. Hill. 1997. Spatial mapping of sedimentary contaminants in the Baltimore Harbor/Patapsco River/Back River system UMCES[CBL]97-142. Maryland Department of the Environment.
5. Bedard, D. L., and J. F. Quensen. 1995. Microbial reductive dechlorination of polychlorinated biphenyls, p. 127-216. In L. Y. Young and C. E. Cemiglia (ed.), Microbial transformation and degradation of toxic organic chemicals. A. John Wiley, New York.
6. Berkaw, M., K. R. Sowers, and H. D. May. 1996. Anaerobic ortho dechlorination of polychlorinated biphenyls by estuarine sediments from Baltimore Harbor. Appl. Environ. Microbiol. 62:2534-2539.
7. Brown, J. J. F., R. E. Wagner, H. Feng, D. L. Bedard, M. J. Brennan, J. C. Carnahan, and R. J. May. 1987. Environmental dechlorination of PCBs. Environ. Toxicol. Chem. 6:579-593.
8. Bunge, M., L. Adrian, A. Kraus, M. Opel, W. G. Lorenz, J. R. Andreesen, H. Görisch, and U. Lechner. 2003. Reductive dehalogenation of chlorinated dioxins by an anaerobic bacterium. Nature 421:357-360.
9. Cochran, W. G. 1950. Estimation of bacterial densities by means of the “Most Probable Number”. Biometrics 6:105-116.
10. Cole, J. R., B. Chai, T. L. Marsh, R. J. Farris, Q. Wang, S. A. Kulam, S. Chandra, D. M. McGarrell, T. M. Schmidt, G. M. Garrity, and J. M. Tiedje. 2003. The Ribosomal Database Project (RDP-II): previewing a new autoaligner that allows regular updates and the new prokaryotic taxonomy. Nuc. Acid. Res. 31:442-443.
11. Connors, T. F., J. F. Rusling, and A. Owlia. 1985. Determination of standard potentials and electron-transfer rates for halobiphenyls from electrocatalytic data. Anal. Chem. 57:170-174.
12. Cupples, A. M., A. M. Spormann, and P. L. McCarty. 2003. Growth of Dehalococcoides-like microorganism on vinyl chloride and cis-dichloroethene as electron acceptors as determined by competitive PCR. Appl. Environ. Microbiol. 69:953-959.
13. Cutter, L., K. R. Sowers, and H. D. May. 1998. Microbial dechlorination of 2,3,5,6-tetrachlorobiphenyl under anaerobic conditions in the absence of soil or sediment. Appl. Environ. Microbiol. 64:2966-2969.
14. Cutter, L. A., J. E. M. Watts, K. R. Sowers, and H. D. May. 2001. Identification of a microorganism that links its growth to the reductive dechlorination of 2,3,5,6-chlorobiphenyl. Environ. Microbiol. 3:699-709.
15. Duhamel, M., K. Mo, and E. A. Edwards. 2004. Characterization of a highly enriched Dehalococcoides-containing culture that grows on vinyl chloride and trichloroethene. Appl. Environ. Microbiol. 70:5538-5545.
16. Edwards, U., T. Rogall, H. Blocker, M. Emde, and E. C. Bottger. 1989. Isolation and direct complete nucleotide determination of entire genes characterization of a gene coding for 16s-ribosomal RNA. Nuc. Acids Res. 19:7843-7853.
17. Felsenstein, J. 1989. PHYLIP—Phylogeny Inference Package (Version 3.2). Cladistics 5:164-166.
18. Fennell, D. E., 1. Nijenhuis, S. F. Wilson, S. H. Zinder, and M. M. Haggblom. 2004. Dehalococcoides ethenogenes strain 195 reductively dechlorinates diverse chlorinated aromatic pollutants. Environ. Sci. Technol. 38:2075-2081.
19. Frame, G. M., J. W. Cochran, and S. S. Bowadt. 1996. Complete PCB congener distributions for 17 Aroclor mixtures determined by 3 HRGC systems optimized for comprehensive, quantitative, congener-specific analysis. J. High Resol. Chromatogr. 19:657-668.
20. Garton, L. S., J. S. Bonner, A. N. Ernest, and R. L. Autenrieth. 1996. Fate and transport of PCBs at the New Bedford Harbor superfund site. Environ. Toxicol. Chem. 15:736-745.
21. He, J., K. M. Ritalahti, K. L. Yang, S. S. Koenigsberg, and F. E. Löffler. 2003. Detoxification of vinyl chloride to ethene coupled to growth of an anaerobic bacterium. Nature 424:62-65.
22. Hendrickson, E. R., J. A. Payne, R. M. Young, M. G. Starr, M. P. Perry, S. Fahnestock, D. E. Ellis, and R. C. Ebersole. 2002. Molecular analysis of Dehalococcoides 16S ribosomal DNA from chloroethene-contaminated sites throughout North America and Europe. Appl. Environ. Microbiol. 68:485-95.
23. Huang, Q. D., and J. F. Rusling. 1995. Formal reduction potentials and redox chemistry of polyhalogenated biphenyls in a bicontinuous microemulsion. Environ. Sci. Technol. 29:98-103.
24. Iannuzzi, T. J., S. L. Huntley, N. L. Bonnevie, B. L. Finley, and R. J. Wenning. 1995. Distribution and possible sources of polychlorinated biphenyls in dated sediments from the Newark Bay estuary, New Jersey. Arch. Environ. Contam. Toxicol. 28:108-117.
25. Janse, I., J. Bok, and G. Zwart. 2004. A simple remedy against artificial double bands in denaturing gradient gel electrophoresis. J. Microbiol Meth. 57:279-281.
26. Lake, J. L., R. J. Pruell, and F. A. Osterman. 1992. An examination of dechlorination processes and pathways in New Bedford Harbor sediments. Mar. Env. Res. 33:31-47.
27. Löffler, F. E., Q. Sun, J. Li, and J. M. Tiedje. 2002. 16S rRNA gene-based detection of tetrachloroethene-dechlorinating Desulfuromonas and Dehalcoccoides species. Appl. Env. Microbiol. 66:1369-1374.
28. Maymo-Gatell, X., Y.-T. Chien, J. M. Gossett, and S. H. Zinder. 1997. Isolation of a bacterium that reductively dechlorinates tetrachloroethene to ethene. Science 276:1568-1571.
29. Morris, P. J., W. W. Mohn, J. F. d. Quensen, J. M. Tiedje, and S. A. Boyd. 1992. Establishment of polychlorinated biphenyl-degrading enrichment culture with predominantly meta dechlorination. Appl. Env. Microbiol. 58:3088-94.
30. Muyzer, G., E. C. de Waal, and A. G. Uitterlinden. 1993. Profiling of complex microbial populations by denaturing gradient gel electrophoresis analysis of PCR amplifeid genes coding for 16S rRNA. Appl. Envir. Microbiol. 59:695-700.
31. Nies, L., and T. M. Vogel. 1991. Identification of the proton source for the microbial reductive dechlorination of 2,3,4,5,6-pentachlorobiphenyl. Appl. Env. Microbiol. 57:2771-2774.
32. Pulliam Holoman, T. R., M. A. Elberson, L. A. Cutter, H. D. May, and K. R. Sowers. 1998. Characterization of a defined 2,3,5,6-tetrachlorobiphenyl-ortho-dechlorinating microbial community by comparative sequence analysis of genes coding for 16S rRNA. Appl. Environ. Microbiol. 64:3359-3367.
33. Quensen, J. F., III, S. A. Boyd, and J. M. Tiedje. 1990. Dechlorination of four commercial polychlorinated biphenyl mixtures (Aroclors) by anaerobic microorganisms from sediments. Appl. Environ. Microbiol 56:2360-2369.
34. Rusling, J. F., and C. L. Miaw. 1989. Kinetic estimation of standard reduction potential of polyhalogenated biphenyls. Environ. Sci. Technol. 23:476-479.
35. Safe, S. 1992. Toxicology, structure-function relationship, and human and environmental health impacts of polychlorinated biphenyls: progress and problems. Environ. Health Perspectives 100:259-268.
36. Saitou, N., and M. Nei. 1987. The neighbor-joining method: a new method for reconstructing phylogenetic trees. Mol. Biol. Evol. 4:406-425.
37. Stone, R. 1992. Swimming against the PCB tide. Science 255:798-799.
38. Stull, J. K., D. J. P. Swift, and A. W. Niedoroda. 1996. Contaminant dispersal on the Palos Verdes continental margin .1. sediments and biota near a major California wastewater discharge. Sci. Total Envir. 179:73-90.
39. Stults, J. R., O. Snoeyenbos-West, B. Methe, D. R. Lovley, and D. P. Chandler. 2001. Application of the 5′ fluorogenic exonuclease assay (TaqMan) for quantitative ribosomal DNA and rRNA analysis in sediments. Appl. Environ. Microbiol. 67:2781-2789.
40. W. Ludwig, O. Strunk, R. Westram, L. Richter, H. Meier, Yadhukumar, A. Buchner, T. Lai, S. Steppi, G. Jobb, W. Förster, I. Brettske, S. Gerber, A. W. Ginhart, O. Gross, S. Grumann, S. Hermann, R. Jost, A. König, T. Liss, R. Lüβmann, M. May, B. Nonhoff, B. Reichel, R. Strehlow, A. Stamatakis, N. Stuckmann, A. Vilbig, M. Lenke, T. Ludwig, A. Bode, and K. Schleifer. 2004. ARB: a software environment for sequence data. Nuc. Acid. Res. 32:1363-1371.
41. Watts, J. E. M., S. K. Fagervold, G. S. Miller, C. E. Milliken, H. D. May, and K. R. Sowers. 2004. Microbial reductive dechlorination of organihalide pollutants in marine environment. Mar. Biotechnol. 6:S378-S383.
42. Watts, J. E. M., S. K. Fagervold, K. R. Sowers, and H. D. May. 2005. A PCR based dpecific assay reveals a population of bacteria within the Chlororflexi associated with the reductive dehalogenation of polychlorinated biphenyls. Microbiology. 151, 2039-2046.
43. Watts, J. E. M., Q. Wu, S. B. Schreier, H. D. May, and K. R. Sowers. 2001. Comparative analyses of PCB dechlorinating communities in enrichment cultures using three different molecular screening techniques. Env. Microbiol. 2:710-719.
44. Wu, Q., K. R. Sowers, and H. D. May. 2000. Establishment of a polychlorinated biphenyl-dechlorinating microbial consortium, specific for doubly flanked chlorines in a defined, sediment-free medium. Appl. Environ. Microbiol. 66:49-53.
45. Wu, Q., J. E. M. Watts, K. R. Sowers, and H. D. May. 2002. Identification of a bacterium that specifically catalyzes the reductive dechlorination of polychlorinated biphenyls with doubly flanked chlorines. Appl. Environ. Microbiol. 68:807-812.
46. Wu, Q. Z., K. R. Sowers, and H. D. May. 1998. Microbial reductive dechlorination of aroclor 1260 in anaerobic slurries of estuarine sediments. Appl Environ Microbiol 64:1052-1058.

What is claimed is:

1. A set of primers for use in a detection assay for detecting PCB dechlorinating organisms in a sample, wherein the set of primers comprises SEQ ID NO: 1 and SEQ ID NO: 2 and any nucleotides sequences that are complementary to same, have more than 98% identity or that hybridize under high stringency conditions of 0.1×SSC, 0.1% SDS at 65° C.; and that hybridizes with 16S rRNA of a bacteria.

2. The set of primers according to claim 1, wherein the set of primers comprises nucleotide sequences consisting of SEQ ID NO: 1 and SEQ ID NO: 2.

3. A method for identifying a PCB dechlorinating bacterial organism comprising: (i) extracting genomic DNA from a bacteria cell suspected of being able to dechlorinate PCB chlorinated compounds; (ii) probing the extracted genomic DNA with at least one probe having a sequence selected from the group consisting of:
(a) SEQ ID NO: 1;
(b) SEQ ID NO: 2;
(c) a nucleotide sequence that is complementary to SEQ ID NO: 1 or SEQ ID NO: 2;
(d) a nucleotide sequence having more than 98% identity with SEQ ID NO: 1 or SEQ ID NO: 2;
(e) a nucleotide sequence that hybridizes under high stringency conditions of 0.1×SSC, 0.1% SDS at 65° C.;
and that hybridizes with 16S rRNA of a bacteria,
under suitable hybridization conditions, wherein the identification of a hybridizable nucleic acid fragment confirms the presence of a bacteria capable of dechlorinating PCB chlorinated compounds.

4. A method for identifying a PCB dechlorinating bacterial organism comprising:
(i) extracting genomic DNA from a bacteria cell suspected of being able to dechlorinate PCB chlorinated compounds;
(ii) probing the extracted genomic DNA with the set of primers according to claim 2, under suitable hybridization conditions, wherein the identification of a hybridizable nucleic acid fragment confirms the presence of a bacteria capable of dechlorinating PCB chlorinated compounds.

5. A method for the dechlorination of PCB chlorinated compounds comprising:
contacting a PCB chlorinated compound with an isolated bacterial organism comprising a sequence consisting of SEQ ID NO: 4 under conditions suitable for PCB dechlorination to occur.

6. A method for separating sub-families of PCB dechlorinating bacterial organisms comprising:
(i) extracting total cellular rRNA from a bacteria cell suspected of being able to dechlorinate PCB chlorinated compounds;
(ii) synthesizing complementary DNA strands to the extracted rRNA using a reverse transcriptase and at least one oligonucleotide primer according to claim 1;
(iii) amplifying the newly generated complementary DNA strands to the extracted rRNA of step (ii) using at least one oligonucleotide primer according to claim 1; and
(iv) separating the amplification products by Denaturing Gradient Gel Electrophoresis.

7. An isolated bacterial organism comprising a 16S ribosomal subunit nucleic acid sequence selected from the group consisting of:
(a) a nucleic acid sequence that has more than 99% identity to a nucleic acid sequence of SEQ ID NO 4 and that hybridizes with 16S rRNA of a bacteria; and
(b) a nucleic acid sequence fully complementary to a nucleic acid of (a); and
wherein the isolated bioremediative microorganism anaerobically dechlorinates chlorinated biphenyls under conditions suitable for PCB dechlorination to occur.

8. A bioremediation composition comprising the isolated bacterial organism according to claim 7 in an amount sufficient to reduce PCB contamination.

9. The bioremediation composition according to claim 8, wherein the composition is mixed with mixed with nutrients and deposited over PCB-contaminated material at sites containing PCBs.

10. The bioremediation composition according to claim 8, wherein the composition is added to a fixed bed, bioreactor, or biofilter for treatment of PCB-contaminated water.

11. A diagnostic nucleic acid gene fusion useful in Denaturing Gradient Gel Electrophoresis (DGGE) having the general structure: SS-GC, wherein:
(i) SS is a signature sequence selected from the group consisting of SEQ ID NO: 1 and SEQ ID NO: 2; and
(ii) GC is a GC clamp sequence having the sequence as set forth in SEQ ID NO: 5.

12. The diagnostic nucleic acid fusion of claim 11 wherein the GC clamp sequence is attached at either 5′ end of the signature sequence.


SEQUENCE LISTING (5 sequences)

SEQ ID NO: 1 - DNA, 17 bp, Artificial Sequence
  Synthetic Construct
gaggcagcagcaaggaa

SEQ ID NO: 2 - DNA, 18 bp, Artificial Sequence
  Synthetic Construct
ggcgggacacttaaagcg

SEQ ID NO: 3 - DNA, 33 bp, Artificial Sequence
  Synthetic Construct
cgcccgccgcgcgcgggaggcagcagcaaggaa

SEQ ID NO: 4 - DNA, 1146 bp, Artificial Sequence
  Synthetic Construct
gtgctttatgcatgcaagttgaacggtcttgatttattaagatagtagcaaacgggtgagtaacacgtaagtaacctgcccctaagcgggggacaacttcgggaaaccgaggctaataccgcatgtgatggtgaaggtaacgcttcattattaaagccttcgggcacttagggaggggctcgcggccgattagctagttggtagggtaacggcttaccaaggctttgatcggtagctggtctgagaggacgatcagccacactgggactgagacacggcccagactcctacgggaggcagcagcaaggaattttgggcaatgggcgaaagcctgacccagcaacgccgcgtgagggatgaaggccttcgggtcgtaaacctcttttctcagggaagaaaaaaatgacggtacctggggaataagtctcggctaactacgtgccagcagccgcggtaatacgtaggaggcgagcgttatccggatttattgggcgtaaagagagcgtaggcggtttgtcaagtcggatgtgaaatctcccggctcaactgggaggagtcattcgatactgatgggctagagtgcagcaggggaaaacggaattcccggtgtagtggtgatatgcgtagataccgggaggaacaccagaggcgaaggcggttttctaggctgtttctgacgctgaggctcaaaagcgtggggagcgaacaggattagataccctggtagtccacgccgtaaacgatggacactaggtatagggagtatcgaccctctctgtgccgaagctaacgctttaagtgtcccgcctggggagtacggccgcaaggctaaaactcaaaggaattgacgggggcccgcacaagcagcggagcgtgtggtttaattcgatgcaacgcgaagaaccttaccaaggtttgacatgtcggaagtagtgacctgaaaaggaaacaacctgttaagtcaggaaccgtcacaggtgctgcatggctgtcgtcagctcgtgccgtgaggtgtttggttaagtcctgcaacgagcgcaaccctcatcgttagttgatttctctagcgagactgccccgcaaaacggggaggaaggtggggatgacgtcaagtcagcatggcccttataccttgggctacacaca

SEQ ID NO: 5 - DNA, 16 bp, Artificial
  Synthetic Construct
cgcccgccgcgcgcgg